(19) United States
(12) Patent Application Publication
(10) Pub. No.: US 2006/0155314 A1
(43) Pub. Date: Jul. 13, 2006

(54) Pathogen resistant carbide surgical tools

(76) Inventors: John C. Pappas III, Southborough, MA (US); Paul N. Pappas, Marlborough, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/525,695

(22) PCT Filed: Aug. 19, 2003
(86) PCT No.: PCT/US03/25903

§ 371 Date: Feb. 22, 2005

Related U.S. Application Data
(60) Provisional application No. 60/404,513, filed on Aug. 19, 2002.

(51) Int. Cl. A61B 17/32

(52) U.S. Cl. 606/167

(57) ABSTRACT

A surgical tool may include a cutting surface having fewer than about 10 pores per square centimeter that are greater than about 15 nanometers, 12 nanometers, 10 nanometers, 5 nanometers, or 5 angstroms in size. The tool may be made of a carbide, such as nickel binder tungsten carbide. The cutting surface may be prepared by polishing it until it has the desired surface porosity.

### RELATED APPLICATIONS

This application claims priority to U.S. Provisional Application 60/404,513 filed on Aug. 19, 2002, which is incorporated in its entirety by reference.

### FIELD OF THE INVENTION

This invention is drawn to surgical tools fabricated from carbide having a surface finish of such limited porosity that corrosion is reduced and entrapment or attachment of pathogens is minimized. In one embodiment the surface porosity of a surgical tool made from carbide is resistant to prion entrapment or attachment.

### BACKGROUND OF INVENTION

Conventional surgical tools having cutting surfaces have been fabricated from a variety of metals using the basic criteria of selecting blade material to be as hard as possible for the job, forming a cutting edge by some mechanical means, such as machining, chipping, etc., hardening the cutting surface and establishing a sharp edge by lapping, honing, sharpening, etc.

Frequently, because hardness and ductility are generally inverse material properties, materials that are less than full-hard are used to provide toughness to the blade. Unfortunately, the integrity of the cutting edge of a surgical tool fabricated from materials of lesser hardness are susceptible to dulling during the surgical procedure.

A surgical cutting edge that maintains its integrity throughout the surgical procedure is highly desirable. A cutting edge that dulls during the course of surgery, unfortunately, causes increased tissue trauma and therefore a prolonged period of healing of an incision and potential scaring or infection as a result of the incision being open for a longer period of time.

Surgical cutting edges produced from materials having a high porosity are prone to corrosion and therefore dulling of the cutting surface due to this corrosion. Additionally, surgical tools fabricated from materials having a high porosity are also prone to pathogen entrapment and or attachment. Contaminated surgical tools will infect a patient during surgery. It has been recently reported that the transmissible agent of Creurzeldt-Jakob disease (CJD) is not readily destroyed by conventional sterilization of surgical instruments. Without being bound by any particular theory, it is believed that the agent responsible for transmissible spongiform encephalopathies, such as CJD, is a prion. A prion is far more resistant to physical and chemical inactivation than conventional pathogens.

More than 100 cases of proven or suspected iatrogenic prion transmissions to humans have been reported. Particular note is made of prion entrapment or binding encountered with stainless steel surfaces in “Infectivity of Scrapie Prions Bound to a Stainless Steel Surface” Molecular Medicine 5; 240-243, (1999), the contents of which are incorporated in their entirety by reference. Without being bound by any particular theory, it is thought that prions have a molecular size of about 35 to 50 angstroms. It is thought that prions bind to stainless steel surfaces in part due to the porosity of the stainless steel surface. Prion removal is hampered by surfaces having a porosity that aids entrapment or attachment and inhibits cleaning. It is therefore desirable to produce a surgical tool having a cutting edge and surfaces in contact with tissue to be fabricated from materials having a sub-ferrous porosity. It is also desirable that cutting surfaces and the tools in their entirety have a high density so that cutting surfaces maintain their integrity and most importantly resists pathogen growth or entrapment.

The tactile feel of a surgical tool is also of great importance. Tools made from of low density materials lack a substantial tactile feel. While certain surgical tools benefit from the lightness of these low density materials, such as stainless steel, in certain surgical applications this low density decreases the tactile feel of the instrument. It is therefore desirable to produce a surgical tool made from a material having a density that not only maintains the integrity of cutting surfaces but produces an instrument having sufficient tactile qualities.

Unfortunately, a surgical tool having the above desired qualities has not been possible in the past because of the choice of material and the construction technique of the prior art.

### SUMMARY OF INVENTION

The present invention addresses the above-identified needs by providing a surgical tool fabricated from nickel binder tungsten carbide (“nickel carbide”) wherein the surgical tool maintains the integrity of the cutting edge for periods that are much longer than prior art tools. The sub-ferrous porosity of nickel carbide advantageously provides a surgical tool that is resistant to corrosion and most importantly resistant to pathogen attachment or entrapment. Additionally, surgical tools fabricated from materials such as nickel carbide produce a tool having superior tactile qualities.

In one illustrative embodiment a non-magnetic surgical tool is fabricated from nickel carbide. This illustrative surgical tool has a body portion having an ergonomic handle. The ergonomic handle is configured from nickel carbide. Nickel carbide has a density of about 14 to about 17 g/cm³ with particular reference to about 15 g/cm³ and a sub-ferrous porosity. The ergonomics of the handle have been optimized to take advantage of weight and balance of nickel carbide.

In some embodiments a surgical tool of this invention has cutting surfaces fabricated from nickel carbide affixed to the body portion of the tool. Carbide cutting surfaces maintain the sharpness of the cutting surface for a longer duration than that of prior art materials. According to the invention, the cutting surface of the tool in total also has a sub-ferrous porosity that prevents corrosion and inhibits pathogen binding or entrapment. This sub-ferrous porosity limits prion binding or entrapment. Without being bound by any particular theory, it is thought that this smooth cutting surface and smooth surgical tools having a sub-ferrous porosity allow for the physical removal of pathogens that are far more resistant to physical and chemical inactivation, such as prions. Additionally, it is thought that pathogens yet unidentified are avoided by surgical instruments fabricated from materials having a sub-ferrous porosity. Furthermore, the non-magnetic nature of a surgical instrument fabricated from nickel carbide allows surgeons the ability to conduct surgery with the concurrent use of diagnostic medical devices dependant upon magnetic radiation, such as nuclear magnetic resonance.

### BRIEF DESCRIPTION OF THE DRAWINGS

The foregoing and other features and advantages of the present invention will be more fully understood from the following detailed description of illustrative embodiments, taken in conjunction with the accompanying drawings in which:

FIG. 1 is a diagrammatic representation of a surgical tool according to the invention being in the form of a chisel having a cutting edge with sub-ferrous surface porosity;

FIG. 2 is a diagrammatic representation of a surgical tool according to the invention being in the form an osteomtome having a cutting edge that is 9 mm wide with sub-ferrous surface porosity;

FIG. 3 is a diagrammatic representation of a surgical tool according to the invention being in the form of an osteomtome having a cutting edge that is 5 mm wide with sub-ferrous surface porosity;

FIG. 4 is a diagrammatic representation of a surgical tool according to the invention being in the form of an osteomtome having a cutting edge that is 2 mm wide with sub-ferrous surface porosity;

FIG. 5 is a diagrammatic representation of a surgical tool according to the invention being in the form of a chisel having a v-shaped cutting edge with sub-ferrous surface porosity;

FIG. 6 is a diagrammatic representation of a surgical tool according to the invention being in the form of an osteotome with a double guard that is 10 mm wide with sub-ferrous surface porosity;

FIG. 7 is a diagrammatic representation of a surgical tool according to the invention being in the form of a guarded osteotome 6 mm wide with sub-ferrous surface porosity;

FIG. 8 is a diagrammatic representation of a surgical tool according to the invention being in the form of a chisel having a curved cutting edge 6 mm wide with sub-ferrous surface porosity;

FIG. 9 is a diagrammatic representation of a surgical tool according to the invention being in the form of a guarded osteotome 6 mm wide curved left with sub-ferrous surface porosity;

FIG. 10 is a diagrammatic representation of a surgical tool according to the invention being in the form of a chisel having a curved cutting edge that is ⅜ inches wide with sub-ferrous surface porosity;

FIG. 11 is a diagrammatic representation of a surgical tool according to the invention being in the form of a chisel having a straight cutting edge that is ⅜ inches wide with sub-ferrous surface porosity;

FIG. 12 is a diagrammatic representation of a surgical tool according to the invention being in the form of a rasp having a cutting edge that is pitched 0.060 with a depth of 0.022 with sub-ferrous surface porosity;

FIG. 13 is a diagrammatic representation of a surgical tool according to the invention being in the form of a rongeur with sub-ferrous surface porosity; and

FIG. 14 is a diagrammatic representation of a surgical tool according to the invention being in the form of a osteotome 2 mm wide with a great handle with sub-ferrous surface porosity.

### DETAILED DESCRIPTION OF THE INVENTION

The present invention relates to surgical tools configured from nickel binder tungsten carbide having a sub-ferrous porosity.

The invention will be better understood with reference to the following definitions:

A. “Porosity” shall mean the incident of voids, gaps or indentations of any sort at any exterior surface wherein said porosity extends below a baseline;

B. “Sub Ferrous Porosity” shall mean having a porosity that is less than that of stainless steel. Particular note is made with surfaces having fewer than about 10 pores/sqcm greater than about 15 nm and in some instances fewer than about 10 pores/sqcm greater than about 10 nm and more particularly fewer than about 10 pores/sqcm greater than about 5 angstroms, and about 50 anstroms;

C. “Density” shall mean a weight of a material per unit volume;

D. “Piron” shall be broadly construed to mean any protein material having a molecular size of approximately 35-50 angstroms and a molecular weight of approximately 33-35 Kda;

E. “Piron Loading” shall mean a concentration of pirons having a sufficient concentration to constitute an infecting dose;

F. “Binding” shall be broadly construed to mean attachment by any means be it chemical, mechanical or electrically charge or mechanical entrapment.

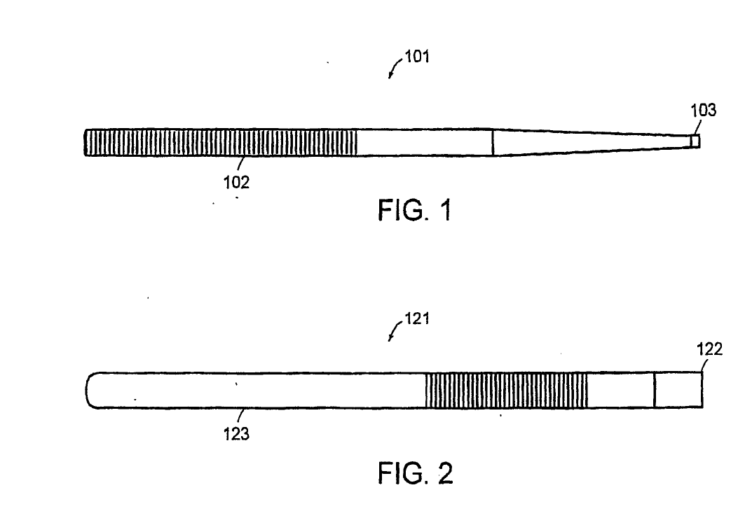

A surgical tool embodying the invention, shown in FIGS. 1-14, comprises two parts a body portion and a cutting surface that are both constructed from a nickel carbide material. In some particular embodiments surgical tools will be comprised of multiple parts. It will be understood that in some specific embodiments, the gauge of the material is chosen so as to provide sufficient flexibility yet preventing deformation of the surgical tool in normal use, and providing the desired spring rate, according to the intended use of the tool.

In some specific embodiments, the body portion of the surgical tool is entirely fabricated from nickel carbide. In other specific embodiments the body portion is a combination of nickel carbide and other materials known in the art. The cutting surfaces according to the invention are fabricated from nickel carbide having a sub-ferrous porosity and a density of about 14 to 17 g/cm³. This sub-ferrous porosity of cutting surfaces will have a tendency to exclude pathogens, such as prions, from attachment or entrapment of any sort.

In one illustrative embodiment nickel carbide having a composition of about 88.5 percent tungsten carbide and about 11.5 percent nickel alloy binder with a density of about 14.3 to 14.9 g/cm³ from Carmet® Company, Royal Oak, Mich. was used to fabricate various illustrative surgical tools. The fabrication of the various illustrative tools was done by using a carbide cutting saw. It is contemplated within the scope of the invention that other methods known in the art of cutting carbide material may be used. The sharpening of the cutting surfaces of the surgical tool was formed using methods known in the art. It is contemplated within the scope of the invention that the porosity of the cutting edge can be further decreased by the use of fine grade carbide particles and adjustment of the hardness of the nickel carbide material.

Prior art surgical tools have been traditionally constructed from stainless steel having a typical density of about 7-8 g/cm³ and a porous surface. These traditional stainless steel tools are cleaned and subjected to a “sterilization” procedure prior to surgical use. It has been recently discovered that certain pathogens survive typical sterilization. Specifically, the transmissible agent of Creurzfeldt-Jakob disease (CJD) is not readily destroyed by conventional sterilization of stainless steel surgical tools, as noted above. The surface of stainless steel surgical tools, while appearing to be smooth and non-porous are in fact, at a microscopic level, porous. Without being bound to any particular theory, it is thought that this porosity accommodates the entrapment or binding of some pathogens and subsequent contamination of patients. Once again, without being bound to any particular theory, this binding may be in the form of mechanical entrapment within the surface pores or it may be in the form of promoting chemical binding and attachment using conventional chemical bonds. For those pathogens, such as prions, that are resistant to sterilization, a high surface porosity allows retention, such as mechanical entrapment of these microscopic pathogens.

Prions typically have a molecular size of approximately 35 to 50 angstroms and a molecular weight of approximately 33-35 Kda. The mechanical entrapment of prions within surface pores of surgical instruments is increased as the surface porosity is increased. This increase in surface porosity causes a surface to achieve a prion loading. This prion loading can be substantially reduced by decreasing the porosity of the surface of a surgical tool.

The surface porosity of a cutting edge or other surfaces areas coming into contact with tissue during a surgical incision must be decreased below about 10-12 nm and preferably below about 50 angstroms to inhibit prion loading. This decrease in prion load reduces subsequent transmission of an infecting prion.

Additionally, the mechanical entrapment of prions and other sub-microscopic size pathogens are greatly reduced and subject to physical removal as the porosity of the surface is decrease. Without being bound by any particular theory, it is thought that decontamination by mechanical means, such as washing, is increased in its effectiveness as the porosity of the surface is decreased. Mechanical washing of the instrument with compounds such as formaldehyde, benzene, ethanol and other compounds known in the art is significantly more effective if porous entrapment surfaces are diminished.

According to the invention, the cutting edge of various surgical tools has been fabricated from a nickel carbide composition reducing the porosity of the cutting surface over that of traditional stainless steel cutting surfaces. It is contemplated within the scope of the invention that other carbides may be used. Carbides such as but not limited to titanium, tantalum, vanadium, zirconium, chromium, hafnium, cerium, manganese, thorium, zirconium and niobium may be used. It is also contemplated within the scope of the invention that binders such as cobalt and nickel may be used. In will be understood by those skilled in the art that various compositions of the above binders and carbides can be used to achieve desired hardness, density and surface porosity. It will also be understood by those skilled in the art that additional special processing, such as hot isostatic pressing, will density the material structure to substantially reduce surface porosity of the carbide material.

In the formation of the cutting edge of the surgical tool, according to the invention, the edge is formed using traditional methods of grinding the carbide material with diamond wheels. The cutting edge is further formed by additional high speed grinding once again using diamond wheels. The sub-ferrous porosity of the cutting edge is achieved by the final finishing of the cutting edge using diamond polishing compound. The cutting edge is honed to a mirror like finish by rubbing the cutting edge with diamond polishing compound.

It has been found that surgical tools fabricated from nickel carbide produce a surgical instrument that is non-magnetic. This non-magnetic quality of the surgical tool is advantageously utilized by surgeons using diagnostic equipment, which require electro magnetic radiation, during surgical procedures. The use of surgical tools according to the invention concurrently with diagnostic equipment such as nuclear magnetic resonance is contemplated with the scope of the invention. It is further contemplated that this non-magnetic nature can be advantageously utilized by surgeons when using multiple surgical tools during a procedure preventing those tools from attraction to each other.

It has also been found that the nickel carbide material having a high density when used within the body of the surgical instrument imparts a tactile feel to the instrument that is not possible with lower density materials. The high density of the carbide material advantageously gives the surgeon a tool having a substantially greater tactile feel then that of lighter weight materials.

According to the invention, various surgical tools having a cutting edge and a body portion are contemplated. Turning to FIG. 1, a diagrammatic representation of a surgical tool according to the invention being in the form of a chisel 101 is shown. The chisel 101 has a handle 102 fabricated from nickel carbide. Affixed to the handle 102 is a cutting edge 103 also fabricated from nickel carbide. The nickel carbide forming the cutting edge 103 has a desired density and sub-ferrous porosity.

Turning to FIG. 2, a diagrammatic representation of a surgical tool according to the invention being in the form of an osteomtome 121 having a cutting edge 122 that is 9 mm wide is shown. The osteomtome 121 has a handle 123 fabricated from nickel carbide. Affixed to the handle 123 is the cutting edge 122 also fabricated from nickel carbide. The nickel carbide forming the cutting edge 122 has a desired density and sub-ferrous porosity.

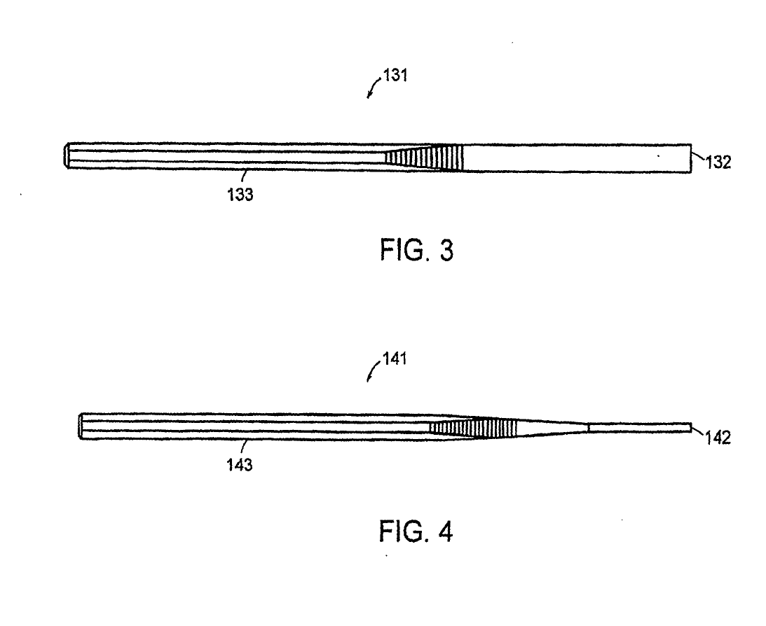

Turning to FIG. 3, a diagrammatic representation of a surgical tool according to the invention being in the form of an osteomtome 131 having a cutting edge 132 that is 5 mm wide is shown. The osteomtome 131 has a handle 133 fabricated from nickel carbide. Affixed to the handle 133 is the cutting edge 132 also fabricated from nickel carbide. The nickel carbide forming the cutting edge 132 has a desired density and sub-ferrous porosity.

Turning to FIG. 4, a diagrammatic representation of a surgical tool according to the invention being in the form of an osteomtome 141 having a cutting edge 142 that is 2 mm wide is shown. The osteomtome 141 has a handle 143 fabricated from nickel carbide. Affixed to the handle 143 is the cutting edge 142 also fabricated from nickel carbide. The nickel carbide forming the cutting edge 142 has a desired density and sub-ferrous porosity.

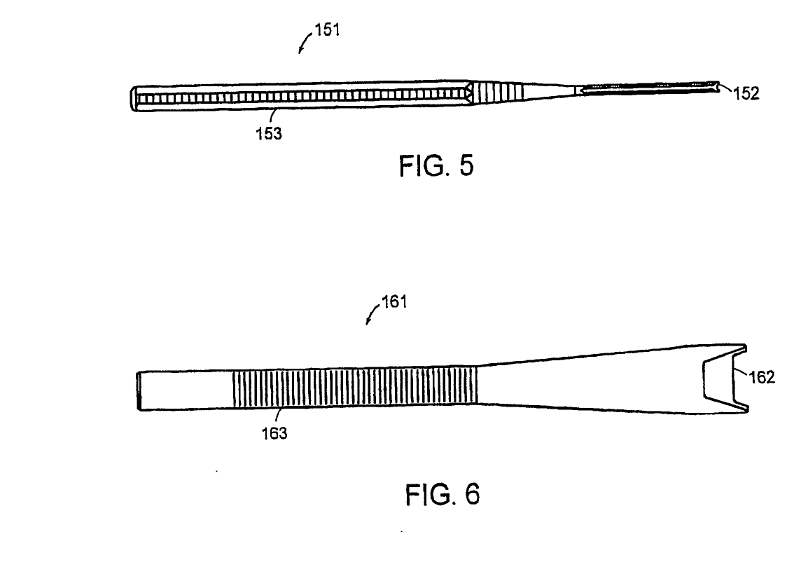

Turning to FIG. 5, a diagrammatic representation of a surgical tool according to the invention being in the form of a chisel 151 having a cutting edge 152 that is v-shaped is shown. The chisel 151 has a handle 153 fabricated from nickel carbide. Affixed to the handle 153 is the cutting edge 152 also fabricated from nickel carbide. The nickel carbide forming the cutting edge 152 has a desired density and sub-ferrous porosity.

Turning to FIG. 6, a diagrammatic representation of a surgical tool according to the invention being in the form of an osteomtome 161 with a double guard that is 10 mm wide having a cutting edge 162 is shown. The osteomtome 161 has a handle 163 fabricated from nickel carbide. Affixed to the handle 163 is the cutting edge 162 also fabricated from nickel carbide. The nickel carbide forming the cutting edge 162 has a desired density and sub-ferrous porosity.

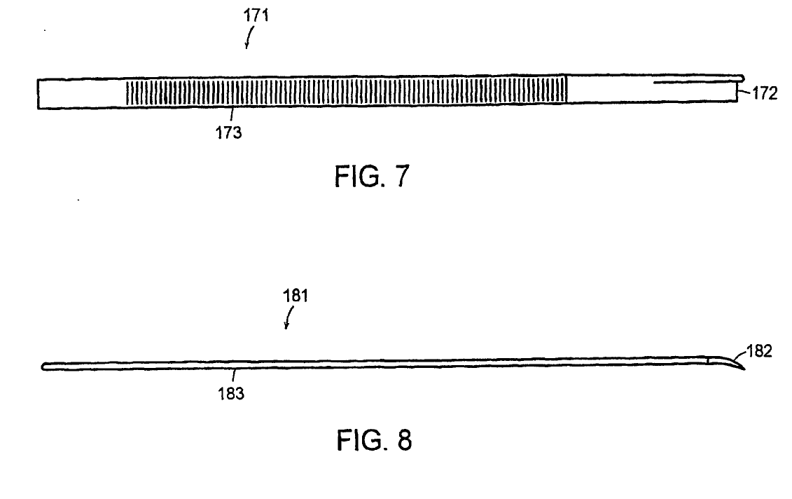

Turning to FIG. 7, a diagrammatic representation of a surgical tool according to the invention being in the form of a guarded osteomtome 6 mm wide 171 having a cutting edge 172 is shown. The osteomtome 171 has a handle 173 fabricated from nickel carbide. Affixed to the handle 173 is the cutting edge 172 also fabricated from nickel carbide. The nickel carbide forming the cutting edge 172 has a desired density and sub-ferrous porosity.

Turning to FIG. 8, a diagrammatic representation of a surgical tool according to the invention being in the form of a chisel 181 having a curved 6 mm cutting edge 152 is shown. The chisel 181 has a handle 183 fabricated from nickel carbide. Affixed to the handle 183 is the cutting edge 182 also fabricated from nickel carbide. The nickel carbide forming the cutting edge 182 has a desired density and sub-ferrous porosity.

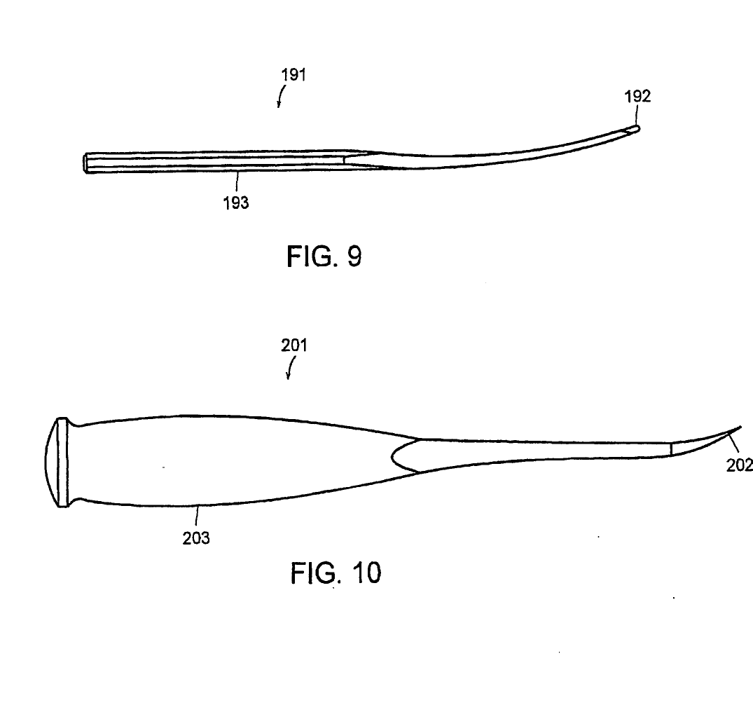

Turning to FIG. 9, a diagrammatic representation of a surgical tool according to the invention being in the form of a guarded osteomtome 6 mm wide curved left 191 having a cutting edge 192 is shown. The osteomtome 191 has a handle 193 fabricated from nickel carbide. Affixed to the handle 193 is the cutting edge 192 also fabricated from nickel carbide. The nickel carbide forming the cutting edge 192 has a desired density and sub-ferrous porosity.

Turning to FIG. 10, a diagrammatic representation of a surgical tool according to the invention being in the form of in the form Chisel 201 having a curved ⅜″ cutting edge 202 is shown. The chisel 201 has a handle 203 fabricated from nickel carbide. Affixed to the handle 203 is the cutting edge 202 also fabricated from nickel carbide. The nickel carbide forming the cutting edge 202 has a desired density and sub-ferrous porosity.

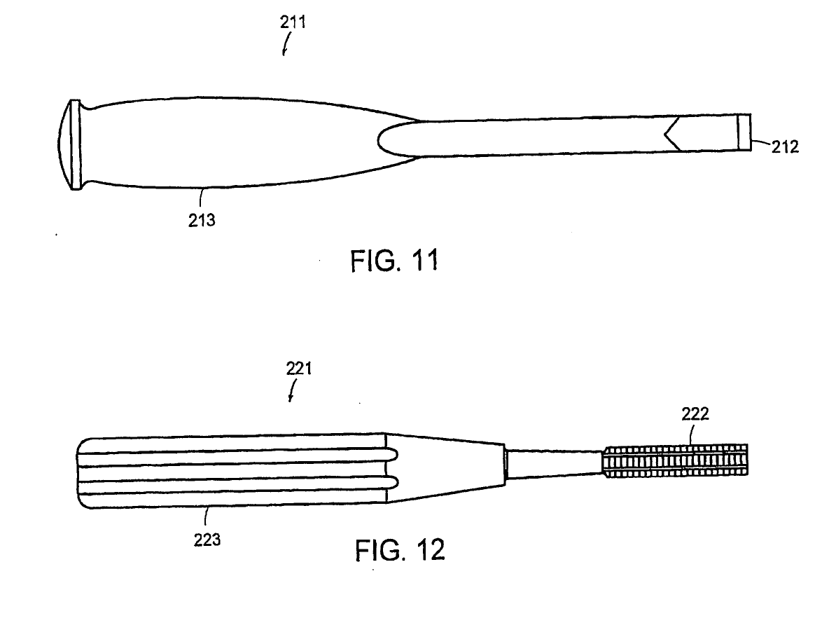

Turning to FIG. 11, a diagrammatic representation of a surgical tool according to the invention being in the form of a chisel 211 having straight cutting edge 212 that is ⅜ inches wide is shown. The chisel 211 has a handle 213 fabricated from nickel carbide. Affixed to the handle 213 is the cutting edge 212 also fabricated from nickel carbide. The nickel carbide forming the cutting edge 212 has a desired density and sub-ferrous porosity.

Turning to FIG. 12, a diagrammatic representation of a surgical tool according to the invention being in the forth of a rasp 221 having a cutting edge 222 that is pitched 0.060 with a depth of 0.22 is shown. The rasp 221 has a handle 223 fabricated from nickel carbide. Affixed to the handle 223 is the cutting edge 222 also fabricated from nickel carbide. The nickel carbide forming the cutting edge 222 has a desired density and sub-ferrous porosity.

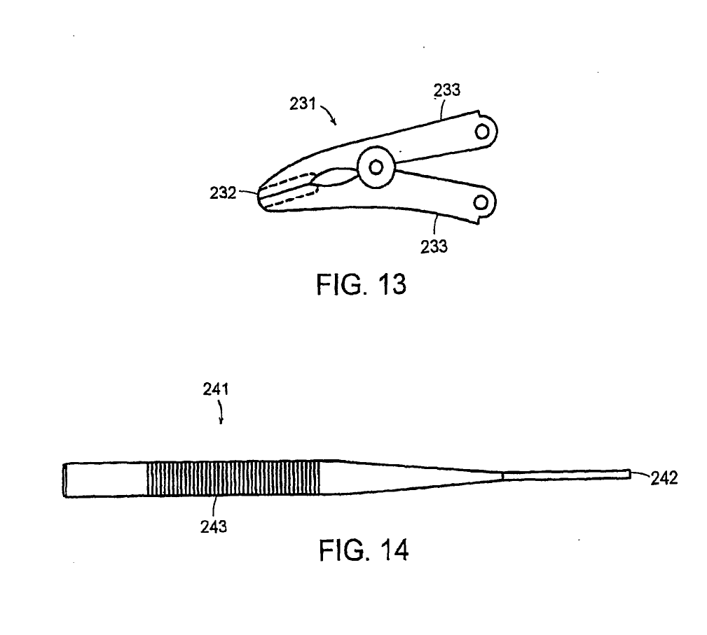

Turning to FIG. 13, a diagrammatic representation of a surgical tool according to the invention being in the form of a rongeur 231 having a cutting edge 232 is shown. The rongeur 231 has a handle 233 fabricated from nickel carbide. Affixed to the handle 233 is the cutting edge 232 also fabricated from nickel carbide. The nickel carbide forming the cutting edge 232 has a desired density and sub-ferrous porosity.

Turning to FIG. 14, a diagrammatic representation of a surgical tool according to the invention being in the form of osteotome 241 having a great handle and a 2 mm cutting edge 242 is shown. The osteotome 241 has a handle 243 fabricated from nickel carbide. Affixed to the handle 243 is the cutting edge 242 also fabricated from nickel carbide. The nickel carbide forming the cutting edge 242 has a desired density and sub-ferrous porosity.

Because tool components are fabricated from nickel carbide having a high density and a low porosity, their manufacture is very straightforward. The surgical tools fabricated from nickel carbide are manufactured using machining methods known in the art. The described surgical tools are easily and thoroughly cleaned and sterilized, having no pores or recesses to harbor contaminants.

While the foregoing describes use of nickel carbide in surgical tools in the field of surgery, the use of nickel carbide may find appropriate uses such at surgical appliances and medical fastening systems requiring a high density low porosity material that is resistant to pathogen growth. In particular it has been found that surgical drills formed from carbide material dissipate heat in a much more efficient manner than that of other materials. This efficient dissipation of heat reduces heat build-up and thus avoids tissue damage caused by excessive heat. Particular reference is made to bone drill bits.

All reference cited within the text of this application are incorporated in their entirety by reference.

While the invention has been described in connection with specific illustrative embodiments thereof, it will be understood that it is capable of further modifications and this application is intended to cover any variations, uses, or alterations of the invention. In general, the principles of the invention and including such departures from the present disclosure as come within known or customary practice within the art to which the invention pertains and as may be applied to the essential features hereinbefore set forth and as follows in the scope of the appended claims.

Various other changes, omissions and additions in the form and detail of the present invention may be made therein without departing from the spirit and scope of the invention.

1-18. (canceled)

19. A surgical tool comprising a cutting surface having fewer than about 10 pores per square centimeter that are greater than about 15 nanometers in size.

20. The surgical tool of claim 19, wherein the cutting surface has no pores that are greater than about 15 nanometers in size.

21. The surgical tool of claim 19, wherein the cutting surface has fewer than about 10 pores per square centimeter that are greater than about 12 nanometers in size.

22. The surgical tool of claim 21, wherein the cutting surface has no pores that are greater than about 12 nanometers in size.

23. The surgical tool of claim 19, wherein the cutting surface has fewer than about 10 pores per square centimeter that are greater than about 10 nanometers in size.

24. The surgical tool of claim 23, wherein the cutting surface has no pores that are greater than about 10 nanometers in size.

25. The surgical tool of claim 19, wherein the cutting surface has fewer than about 10 pores per square centimeter that are greater than about 5 nanometers in size.

26. The surgical tool of claim 25, wherein the cutting surface has no pores that are greater than about 5 nanometers in size.

27. The surgical tool of claim 19, wherein the cutting surface has fewer than about 10 pores per square centimeter that are greater than about 5 angstroms in size.

28. The surgical tool of claim 19, wherein the cutting surface comprises carbide.

29. The surgical tool of claim 28, wherein the cutting surface comprises nickel binder carbide.

30. The surgical tool of claim 28, wherein the cutting surface comprises tungsten carbide.

31. The surgical tool of claim 30, wherein the cutting surface comprises nickel binder tungsten carbide.

32. The surgical tool of claim 31, wherein the cutting surface comprises nickel binder tungsten carbide having a composition of about 88.5 percent tungsten carbide and about 11.5 percent nickel alloy binder.

33. The surgical tool of claim 32, wherein the cutting surface has fewer than about 10 pores per square centimeter that are greater than about 12 nanometers in size.

34. The surgical tool of claim 32, wherein the cutting surface has fewer than about 10 pores per square centimeter that are greater than about 10 nanometers in size.

35. The surgical tool of claim 32, wherein the cutting surface has fewer than about 10 pores per square centimeter that are greater than about 5 nanometers in size.

36. The surgical tool of claim 32, wherein the cutting surface has fewer than about 10 pores per square centimeter that are greater than about 5 angstroms in size.

37. The surgical tool of claim 32, wherein the cutting surface comprises at least one of titanium carbide, tantalum carbide, vanadium carbide, zirconium carbide, hafnium carbide, cerium carbide, manganese carbide, thorium carbide, and niobium carbide.

38. The surgical tool of claim 32, wherein the cutting surface comprises cobalt binder carbide.

39. The surgical tool of claim 19, wherein the cutting surface comprises a material having a density of at least about 14 g/cm³.

40. The surgical tool of claim 39, wherein the cutting surface comprises a material having a density of about 14 g/cm³ to about 17 g/cm³.

41. The surgical tool of claim 40, wherein the cutting surface comprises a material having a density of about 15 g/cm³.

42. The surgical tool of claim 40, wherein the cutting surface comprises a material having a density of about 14.3 to 14.9 g/cm³.

43. The surgical tool of claim 19, further comprising a body portion to which the cutting surface is affixed.

44. The surgical tool of claim 43, wherein the cutting surface and the body portion are made of different materials.

45. The surgical tool of claim 44, wherein the cutting surface comprises tungsten carbide.

46. The surgical tool of claim 45, wherein the cutting surface comprises nickel binder tungsten carbide.

47. The surgical tool of claim 19, wherein the cutting surface is integrally formed with a body portion.

48. The surgical tool of claim 47, wherein the cutting surface and the body portion are made of tungsten carbide.

49. The surgical tool of claim 48, wherein the cutting surface and the body portion are made of nickel binder tungsten carbide.

50. A surgical tool comprising:
a body portion; and
a cutting surface affixed to the body portion, the cutting surface being made of nickel binder tungsten carbide having a density of at least 14 g/cm³, and the cutting surface having fewer than about 10 pores per square centimeter that are greater than about 5 nanometers in size.

51. A surgical tool comprising:
a body portion made of nickel binder tungsten carbide having a density of at least 14 g/cm³; and
a cutting surface integrally formed with the body portion, the cutting surface being made of nickel binder tungsten carbide having a density of at least 14 g/cm³, and the cutting surface having fewer than about 10 pores per square centimeter that are greater than about 5 nanometers in size.

52. A method of making the surgical tool defined by claim 19, comprising:
machining the cutting surface; and
polishing the cutting surface until it has fewer than about 10 pores per square centimeter that are greater than about 15 nanometers in size.

53. The method of claim 52, wherein the cutting surface comprises carbide, and the machining step comprises grinding the carbide with a diamond wheel.

54. The method of claim 53, wherein the polishing step comprises rubbing the cutting surface with diamond polishing compound.

55. The method of claim 54, wherein the polishing step comprises honing the cutting surface to a mirror-like finish.

56. The method of claim 55, further comprising affixing the cutting surface to a body portion of the surgical tool.

57. The method of claim 52, wherein the polishing step comprises honing the cutting surface to a mirror-like finish.

58. A method of making the surgical tool defined by claim 19, comprising processing the cutting edge with hot isostatic pressing so that it has fewer than about 10 pores per square centimeter that are greater than about 15 nanometers in size.

59. A method of making the surgical tool defined by claim 19, comprising forming the cutting edge with fine grade carbide particles.

60. A method of making the surgical tool defined by claim 43, comprising affixing the cutting surface to a body portion of the surgical tool.
